(19) United States
(12) Patent Application Publication
(10) Pub. No.: US 2005/0182106 A1
(43) Pub. Date: Aug. 18, 2005

(54) Medicinal composition for mitigating blood lipid or lowering blood homocysteine

(75) Inventors: Tatsuhito Kondo, Tokyo (JP); Ikuo Takagi, Matsudo-shi (JP); Masato Nakayama, Kitakatsushika-gun (JP); Yasuhiro Torizumi, Ryugasaki-shi (JP)

Correspondence Address: FRISHAUF, HOLTZ, GOODMAN & CHICK, PC, 220 5TH AVE FL 16, NEW YORK, NY, 10001-7708, US

(73) Assignee: SANKYO COMPANY, LIMITED, Tokyo (JP)

(21) Appl. No.: 11/031,105

(22) Filed: Jan. 7, 2005

Related U.S. Application Data
(63) Continuation-in-part of application No. PCT/JP03/08674, filed on Jul. 8, 2003.

(30) Foreign Application Priority Data
Jul. 11, 2002 (JP) 2002-202121
Nov. 27, 2002 (JP) 2002-343586

(51) Int. Cl. A61K 31/4415; A61K 31/401; A61K 31/366; A61K 31/225

(52) U.S. Cl. 514/350; 514/423; 514/460; 514/548

(57) ABSTRACT

A medicinal composition for mitigating blood lipid and for reducing the amount of blood homocysteine. The medicinal composition contains an HMG-CoA reductase inhibitor and a pyridoxine compound.

This is a Continuation-in-Part application of International Application PCT/JP2003/008674 filed on Jul. 8, 2003, incorporated herein by reference in its entirety.

### BACKGROUND OF THE INVENTION

The present invention relates to a medicinal composition comprising an HMG-CoA reductase inhibitor and a pyridoxine derivative as active ingredients (particularly a medicinal composition for mitigating blood lipid levels or lowering high blood homocysteine levels).

An old proverb wisely says that a man is as old as his arteries. Recently, it has been noted and become widely known that increased blood homocysteine levels are an independent risk factor for arteriosclerosis.

Homocysteine is an amino acid produced in the metabolism of methionine, which is one of the essential amino acids. It has been observed that blood homocysteine levels are increased by genetic factors, deficiencies in vitamin cofactors (folic acid, vitamin B6, and vitamin B12), aging, sex difference, renal hypofunction, diabetes mellitus and other diseases, some drugs, smoking, and the like (Progress in Medicine, vol. 19 No. 8, 1999, p. 49-52). This symptom is called homocysteinemia. Treatment against homocysteinemia currently consists of folic acid to advance homocysteine metabolism as a first step, followed by treatment with vitamins B6 and B12 as second-step therapy (Progress in Medicine, vol. 19 No.8, 1999, p. 52-53).

On the other hand statins, which reduce blood cholesterol levels by specifically and competitively inhibiting HMG-CoA reductase in vivo, have elicited the following effects on blood homocysteine levels:
1) Following administration of pravastatin for 8 successive weeks in 16 patients with hypercholesterolemia, blood homocysteine levels were reduced (Nikkei Medical, November 2001, p. 73),
2) In 20,705 patients treated with lovastatin for one year, blood homocysteine levels decreased by 3.7% on average (versus a 1.9% decrease in the placebo control group) (Circulation, vol. 105, No. 15, 2002, p. 1778),
3) Three of 7 patients with cardiovascular diseases were treated with atorvastatin and the other 4 patients were treated with simvastatin for 6 successive weeks. Blood homocysteine levels in patients treated with atorvastatin were increased, while those in patients treated with simvastatin were decreased (31st Hemophilia Symposium Hamburg, 2000, p. 258).

Thus it would appear that the results are inconsistent and no clear conclusion has yet been reached.

Furthermore, combination therapy with statins plus folic acid has been disclosed to prevent or reduce the risk of developing arteriosclerosis (WO 97/38694).

Regarding combination therapy with a statin plus a pyridoxine derivative, it has also been described on page 17 of WO 97/38694 that some other agents selected from HMG-CoA synthetase inhibitors, squalene epoxidase inhibitors, squalene synthetase inhibitors, ACAT inhibitors, probucol, niacin, fibrates, cholesterol-binding antagonists, bile acid sequestrants, LDL receptor inducers, vitamin B6, vitamin B12, aspirin, β-blockers, vitamin C, vitamin E, and β-carotene may be contained in the combination therapy in addition to a statin and folic acid.

However, there is no description in WO 97/38694 of a combination therapy containing an HMG-CoA synthetase inhibitor and a pyridoxine derivative alone, which is the present invention. Furthermore, there are neither descriptions nor suggestions about remarkable mitigating effects on blood lipid levels or reducing blood homocysteine levels, which is the medical intention of the present invention.

On the other hand, a composition comprising an HMG-CoA reductase inhibitor (statin) and 7 supplements (omega-3 fatty acid, vitamin E, vitamin C, vitamin B6, vitamin B12, folic acid, and calcium) is disclosed in WO 02/43659 as a dietary supplement to reduce risk of cardiovascular diseases. However, this composition was contrived from speculation based on the known efficacies of the various individual compositions, that is, statin and omega-3 fatty acid mitigate serum lipid levels, vitamin E and vitamin C exert anti-oxidant activities, a combination containing folic acid, vitamin B6, and vitamin B12 reduces blood homocysteine levels, and calcium is beneficial for the cardiovascular system due to decreases in blood pressure. No concrete data are disclosed in WO 02/43659. Therefore the novel effects of the present invention, namely, the remarkable mitigating effects of the present invention on blood lipid levels and remarkable reducing effects on blood homocysteine levels are not described in WO 02/43659.

### BRIEF DESCRIPTION OF THE INVENTION

In light of this background, the present inventors have diligently conducted research to discover new and safe drugs that mitigate blood lipid levels and reduce blood homocysteine levels, and found that a medicinal Composition comprising an HMG-CoA reductase inhibitor and a pyridoxine derivative as active ingredients exerts good effects on mitigating blood lipids levels and reducing blood homocysteine levels, and thus completed the present invention.

The present invention relates to
(1) a medicinal composition comprising an HMG-CoA reductase inhibitor and a pyridoxine derivative as active ingredients of the said description, the present invention includes
(2) a medicinal composition as stated in (1) for mitigating blood lipid levels or reducing high blood homocysteine levels,
(3) a medicinal composition as stated in (1) or (2), comprising one or more HMG-CoA reductase inhibitors selected from the group consisting of pravastatin, lovastatin, simvastatin, fluvastatin, rivastatin, atorvastatin, pitavastatin, and rosuvastatin as active ingredients,
(4) a medicinal composition as stated in (1) or (2), comprising one or more HMG-CoA reductase inhibitors selected from the group consisting of pravastatin, simvastatin, and atorvastatin as active ingredients,
(5) a medicinal composition as stated in (1) or (2), comprising an HMG-CoA reductase inhibitor of atorvastatin, as an active ingredient,
(6) a medicinal composition according to any one selected from (1) to (5) described above, comprising one or more pyridoxine derivatives selected from the group consisting of pyridoxine, pyridoxal, pyridoxamine, and pharmacologically acceptable salts thereof,
(7) a medicinal composition according to any one selected from (1) to (5) described above, comprising a pyridoxine derivative selected from pyridoxine and a pharmacologically acceptable salt thereof,
(8) a medicinal composition as stated in (2) where the HMG-CoA reductase inhibitor is atorvastatin or a pharmacologically acceptable salt thereof, and the pyridoxamine derivative is pyridoxine or a pharmacologically acceptable salt thereof,
(9) a medicinal composition according to any one selected from (1) to (8) for mitigating blood lipid levels,
(10) a medicinal composition according to any one selected from (1) to (8) for reducing high blood homocysteine levels,
(11) a medicinal composition according to any one selected from (1) to (8) for therapy or prevention of diseases caused by high blood homocysteine levels,
(12) a medicinal composition according to any one selected from (1) to (8) for therapy or prevention of hypercholesterolemia, arteriosclerosis, ischemic heart disease, myocardial infarction, thrombosis, peripheral vascular diseases, Burger's disease, Raynaud's disease, cerebral infarction, cerebrovascular disorders, senile dementia, Alzheimer's disease, or Parkinson disease, and
(13) a medicinal composition as stated in (8) for prevention or therapy of hypercholesterolemia, arteriosclerosis, ischemic heart disease, myocardial infarction, thrombosis, peripheral vascular diseases, Burger's disease, Raynaud's disease, cerebral infarction, cerebrovascular disorders, senile dementia, Alzheimer's disease, or Parkinson disease.

In addition, the present invention provides
(14) combination use of an HMG-CoA reductase inhibitor and a pyridoxine derivative for mitigating blood lipid levels or reducing high blood homocysteine levels by administration of an HMG-CoA reductase inhibitor and a pyridoxine derivative at the same time or independently at certain time intervals, and
(15) combination use of an HMG-CoA reductase inhibitor and a pyridoxine derivative for mitigating blood lipid levels or reducing high blood homocysteine levels.

Furthermore, the present invention provides
(16) a method to mitigate blood lipid levels or to reduce high blood homocysteine levels by administration of an HMG-CoA reductase inhibitor and a pyridoxine derivative at the same time or independently at certain time intervals.

The preferable methods in said (16) are
(17) a method as stated in (16) for mitigating blood lipid levels,
(18) a method as stated in (16) for reducing a high blood homocysteine levels,
(19) a method as stated in (16) for therapy or prevention of diseases caused by high blood homocysteine levels,
(20) a method as stated in (16) for prevention or therapy of hypercholesterolemia, arteriosclerosis, ischemic heart disease, myocardial infarction, thrombosis, peripheral vascular diseases, Burger's disease, Raynaud's disease, cerebral infarction, cerebrovascular disorders, senile dementia, Alzheimer's disease, or Parkinson disease,
(21) a method according to any one selected from (16)-(20), in which a medicinal composition comprising an HMG-CoA reductase inhibitor and a pyridoxine derivative is administered, and
(22) a method as stated in (20), in which a medicinal composition comprising an HMG-CoA reductase inhibitor and a pyridoxine derivative is administered.

Furthermore, the present invention provides
(23) the use of an HMG-CoA reductase inhibitor and a pyridoxine derivative in the manufacture of a medicinal composition comprising an HMG-CoA reductase inhibitor and a pyridoxine derivative for mitigating blood lipid levels or reducing high blood homocysteine levels.

### DETAILED DESCRIPTION OF THE INVENTION

“HMG-CoA reductase inhibitor”, one component of the medicinal composition of the present invention, refers to agents that competitively and specifically inhibit 3-hydroxy-3-methyl-glutaryl-CoA (HMG-CoA) reductase, which is a rate limiting enzyme in the biosynthesis of cholesterol. Since such inhibitors suppress blood cholesterol levels, the inhibitors are used as therapeutic agents for patients with hypercholesterolemia. As such HMG-CoA reductase inhibitors, natural products derived from microorganisms and semi-synthesized compounds derived from the natural products described above, and totally synthesized chemical compounds are all included. For instance, such compounds are (+)-(3R, 5R)-3,5-dihydroxy-7-[(1S,2S,6S,8S,8aR)-6-hydroxy-2-methyl-8-[(S)-2-methylbutyryloxy]-1,2,6,7,8,8a-hexahydro-1-naphtyl]heptanoic acid which is disclosed in Japanese Patent Publication (Kokai) Number Sho 57-2240 (U.S. Pat. No. 4,346,227) (hereinafter called pravastatin), (+)-(1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthyl(S)-2-methylbutyrate which is disclosed in Japanese Patent Publication (Kokai) Number Sho 57-163374 (U.S. Pat. No. 4,231,938) (hereinafter called lovastatin), (+)-(1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthyl 2,2-dimethylbutyrate which is disclosed in Japanese Patent Publication (Kokai) Number Sho 56-122375 (U.S. Pat. No. 4,444,784) (hereinafter called simvastatin), (±)(3R*,5S*,6E)-7-[3-(4-fluorophenyl)-1-(1-methylethyl)-1H-indol-2-yl]-3,5-dihydroxy-6-heptenoic acid which is disclosed in Japanese Patent Publication (Kohyo) Number Sho 60-500015 (U.S. Pat. No. 4,739,073) (hereinafter called fluvastatin), (3R,5S,6E)-7-[4-(4-fluorophenyl)-2,6-di(1-methylethyl)-5-methoxy methylpyridin-3-yl]-3,5-dihydroxy-6-heptenoic acid which is disclosed in Japanese Patent Publication (Kokai) Number Hei 1-216974 (U.S. Pat. No. 5,006,530) (hereinafter called rivastatin), (3R,5S)-7-(2-(4-fluorophenyl)-5-(1-methylethyl)-3-phenyl-4-phenylaminocarbonyl-1H-pyrrol-1-yl]-3,5-dihydroxyheptanoic acid which is disclosed in Japanese Patent Publication (Kokai) Number Hei 3-58967 (U.S. Pat. No. 5,273,995) (hereinafter called atorvastatin), and (E)-3,5-dihydroxy-7-[4′-(4′-fluorophenyl)-2′-cyclopropyl-quinolin-3′-yl]-6-heptenoic acid which is disclosed in Japanese Patent Publication (Kokai) Number Hei 1-279866 (U.S. Pat. No. 5,854,259 and U.S. Pat. No. 5,856,336) (hereinafter called pitavastatin), or (+)-(3R,5S)-7-[4-(4-fluorophenyl)-6-isopropyl-2-(N-methyl-N-methanesulfonylamino)pyridin-5-yl]-3,5-dihydroxy-6(E)-heptenoic acid which is disclosed in Japanese Patent Publication (Kokai) Number Hei 5-178841 (U.S. Pat. No. 5,260,440) (hereinafter called rosuvastatin). In addition, an HMG-CoA reductase inhibitor, which is one component of the medicinal composition of the present invention, refers to other HMG-CoA reductase inhibitors described in the disclosed patents described above.

Planar chemical structures of representative HMG-CoA reductase inhibitors are shown below:

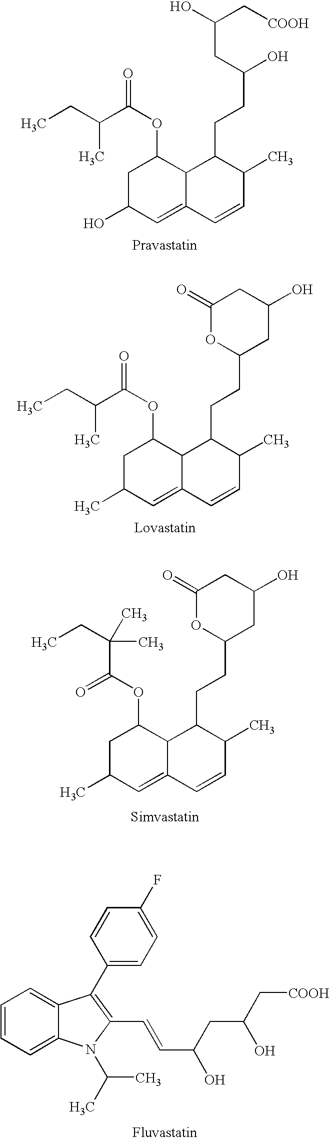

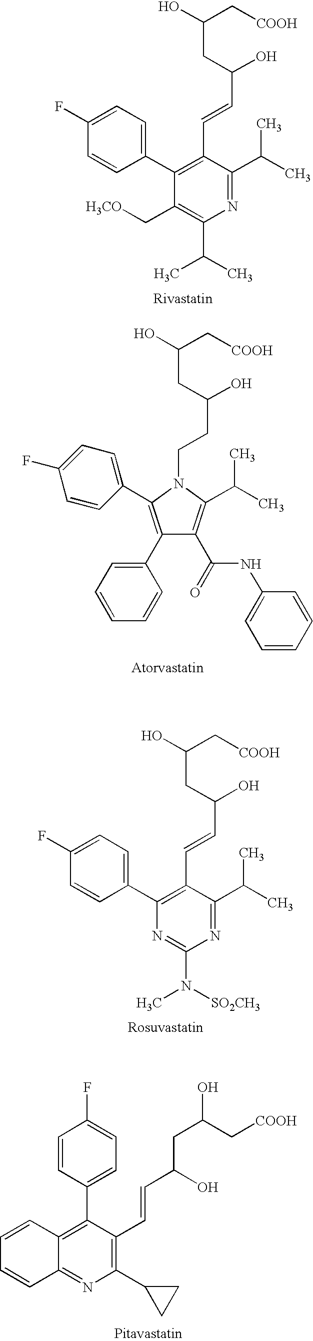

“Pyridoxine derivative”, which is an active ingredient of the medicinal composition of the present invention includes pyridoxine, pyridoxal, pyridoxamine, or salts thereof, and is preferably pyridoxine hydrochloride, pyridoxal phosphate, or pyridoxamine phosphate, and is more preferably pyridoxine hydrochloride.

Each active ingredient of the present invention described above may be present as pharmacologically acceptable salts thereof, and
in the case that the active ingredients present a basic functional group, such salts are, for example, a hydrohalide such as hydrofluoride, hydrochloride, hydrobromide, hydroiodide, or the like; an inorganic acid salt such as a nitrate, a perchlorate, a sulfate, a phosphate, or the like; a lower organic sulfonate such as methanesulfonate, trifluoromethanesulfonate, ethanesulfonate, or the like; an arylsulfonate such as benzenesulfonate p-toluenesulfonate, or the like; an amino acid salt such as ornithine salt, glutamate, or the like; carboxylic acid salt such as a fumarate, a succinate, a citrate, a tartrate, an oxalate, a maleate, or the like,
in the case that the active ingredients present an acidic functional group, such salts are, for example, an alkaline metal salt such as sodium salt, potassium salt, lithium salt, or the like; an alkaline earth metal salt such as calcium salt, magnesium salt, or the like; a metal salt such as an aluminium salt, an iron salt, a zinc salt, a copper salt, a nickel salt, a cobalt salt, or the like; an amine salt, for example, an inorganic amine salt such as ammonium salt, an organic amine salt such as t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzyl-phenethylamine salt, piperazine salt, tetramethylammonium salt, tris(hydroxymethyl)aminomethane salt, or the like. For instance, in the case of pravastatin, a preferable salt is pravastatin sodium, and for instance, in the case of atorvastatin, a preferable salt is atorvastatin calcium salt hydrate.

In the case that each active ingredient involved forms a hydrate or solvate, such hydrates or solvates are included in the medicinal compositions of the present invention.

In the present invention, “mitigating blood lipid levels” means reducing blood lipid levels to clinically significant values, namely, reducing blood triglyceride levels, reducing blood LDL levels, or reducing blood total cholesterol levels.

In the present invention, “reducing blood homocysteine levels” means suppressing increases in high blood homocysteine levels, and reducing blood homocysteine levels. Factors that increase homocysteine levels in the blood are, for example, aging, smoking, nutrition impairment related to homocysteine metabolism, some drugs, renal hypofunction, chronic renal dysfunction, diabetes mellitus, insulin resistance, malignant neoplasm, thyroidal hypofunction, pernicious anemia, and the like.

“Diseases caused by high blood homocysteine levels” described in the present invention have no limitation so long as the diseases derive from high blood homocysteine levels. They are, for example, cardiovascular diseases such as arteriosclerosis, ischemic heart disease, myocardial infarction, thrombosis, peripheral vascular disease, Burger's disease, Raynaud's disease, and cerebrovascular diseases such as cerebral infarction, cerebrovascular disorders, senile dementia, neurological diseases such as Alzheimer's disease, Parkinson disease, and the like.

Since the early stages of the said diseases exhibit few subjective symptoms, it is difficult for patients themselves to become aware of having these diseases in the early stages. The subjective symptoms of the “diseases caused by high blood homocysteine levels” in the present invention are, for example, headache, migraine, dizziness, numbness or feeling of numbness, cold sensation of the four limbs, shoulder stiffness, and the like. Therefore the said diseases may possibly be treated at their early stages by taking the medicinal composition of the present invention when such subjective symptoms first appear.

HMG-CoA reductase inhibitors used as an active ingredient in the medicinal composition of the present invention, for example, pravastatin, lovastatin, simvastatin, fluvastatin, rivastatin, atorvastatin, pitavastatin, or rosuvastatin, can be easily prepared according to the methods described hereinafter in Japanese Patent Publication (Kokai) Number Sho 57-2240 (U.S. Pat. No. 4,346,227), Japanese Patent Publication (Kokai) Number Sho 57-16337 (U.S. Pat. No. 4,231,938), Japanese Patent Publication (Kokai) Number Sho 56-122375 (U.S. Pat. No. 4,444,784), Japanese Patent Publication (Kohyo) Number Sho 60-500015 (U.S. Pat. No. 4,739,073), Japanese Patent Publication (Kokai) Number Hei 1-216974 (U.S. Pat. No. 5,006,530), Japanese Patent Publication (Kokai) Number Hei 3-58967 (U.S. Pat. No. 5,273,995), Japanese Patent Publication (Kokai) Number Hei 1-279866 (U.S. Pat. No. 5,854,259and U.S. Pat. No. 5,856,336), or Japanese Patent Publication (Kokai) Number Hei 5-178841 (U.S. Pat. No. 5,260,440).

In addition, pyridoxine derivatives used in the medicinal compositions of the present invention as an active ingredient can be easily obtained as commercially available products, or can be easily manufactured by previously known methods. For example, since pyridoxine hydrochloride is disclosed in “The Japanese Pharmacopoeia (JP) 14th Edition”, the compound can be easily obtained.

The medicinal compositions of the present invention comprising an HMG-CoA reductase inhibitor and a pyridoxine derivative as active ingredients contain both an HMG-CoA reductase inhibitor and a pyridoxine derivative as essential active ingredients. Additive agents may be contained in the formulation, when required. In addition, other inactive ingredients included in the medicinal compositions are not particularly restricted provided that they have no adverse effects when they are co-administered with the HMG-CoA reductase inhibitor and the pyridoxine derivative contained in the present medicinal composition. The preferable medicinal composition is restricted to an HMG-CoA reductase inhibitor and a pyridoxine derivative alone as active ingredients, and it may contain additive agents for its formulation.

The concrete preparations of the medicinal composition of the present invention are, for example, tablets, granules (including powders), capsules, liquids and solutions (including syrups), and the like. These preparations are prepared by conventionally known methods disclosed in “The Japanese Pharmacopoeia (JP)” or the like using additive agents and bases that are suitable for each preparation, as necessary.

In each preparation, various conventionally used additive agents suitable for each preparation may also be used.

For example, in the case of tablets, diluents such as lactose, crystalline cellulose or the like, stabilizers such as magnesium aluminometasilicate, magnesium oxide or the like, coating agents such as hydroxypropylcellulose or the like, and lubricants such as magnesium stearate or the like may be used.

In the case of granules and capsules, diluents such as lactose, purified sucrose, or the like, stabilizers such as magnesium aluminometasilicate, magnesium oxide, or the like, adsorbents such as corn starch, or the like, binders such as hydroxypropylcellulose, or the like may be used.

In each preparation described above, disintegrants such as crospovidone, or the like; surfactants such as polysorbate, or the like; adsorbents such as calcium silicate, or the like; colouring agents such as red ferric oxide, caramel, or the like; stabilizers such as sodium benzoate, or the like; pH modifiers; flavours; or the like, may be added as necessary.

In the present invention, “co-administration” means methods of administration of more than the 2 active ingredients to humans at the same time, or independent administration of more than the 2 active ingredients described above at a certain time interval.

When the 2 active ingredients of the present invention are administered, each active ingredient of the medicinal composition may be administered at the same time or independently at a certain time interval.

“Administration at the same time” described above includes administration of each active ingredient at a pharmacologically acceptable time interval in addition to administration of all active ingredients at the same time. There is no restriction provided that their pharmaceutical preparations are to be taken at roughly the same time. Nevertheless, it is favourable to take the 2 active ingredients as a single pharmaceutical preparation.

“Independent administration of the more than 2 active ingredients described above at a certain time interval” described above has no restriction provided that their available pharmaceutical preparations are to be taken independently at different times. For instance, it indicates that first one active ingredient is administered, and then after a defined time delay, the other active ingredient is administered.

Furthermore, in the case that the medicinal composition contains more than 3 active ingredients, “simultaneous administration or independent administration at certain time intervals” includes all cases wherein all active ingredients contained in the medicinal composition are taken at the same time, each composition is taken independently at certain time intervals, more than 2 active ingredients contained in the medicinal composition are simultaneously taken and the rest of them are independently taken at certain time intervals, or more than 2 active ingredients in the medicinal composition are simultaneously taken and the rest of them are simultaneously taken at a certain time interval.

Since the medicinal composition of the present invention exerts remarkable mitigating effects on blood lipid levels and reducing effects on high blood homocysteine levels, the medicinal compositions of the present invention are useful as pharmaceutical agents for mitigating blood lipid levels and reducing high blood homocysteine levels. Thus the medicinal compositions of the present invention are useful as preventive or therapeutic agents against diseases, for example, hypercholesterolemia, arteriosclerosis, ischemic heart disease, myocardial infarction, thrombosis, peripheral vascular disease, Burger's disease, Raynaud's disease, cerebral infarction, cerebrovascular disorders, senile dementia, Alzheimer's disease, or Parkinson disease.

In the present invention, the dosage of HMG-CoA reductase inhibitors varies depending on the types of HMG-CoA reductase inhibitors used, the formulations, and the like. It is usual to administer 0.02 mg-3 mg/kg per day and preferably 0.08 mg-2.5 mg/kg per day. For example, in an adult human (about 60 kg), this is a dosage of approximately 1-200 mg per day preferably 5-160 mg per day.

In the present invention, the dosage of pyridoxine derivative varies depending on the type of pyridoxine derivative used, its formulation, and the like. It is usual to administer 0.002-20 mg/kg per day and preferably 0.02-13 mg/kg per day. For example, in an adult human (about 60 kg) this is a dosage of about 0.1 mg-1,200 mg per day and preferably about 1 mg-800 mg per day.

In the case that the dosage form of the medicinal composition of the present invention is a solid dosage form, the weight percentages of the active ingredients contained in the medicinal composition are, for example, usually 0.01-5% in the case of atorvastatin or pravastatin, and preferably 0.05-3%. In the case of simvastatin, the weight percentage is usually 0.005-3%, preferably 0.03-2%. Furthermore, weight percentage of pyridoxine derivative is usually 0.01-30%, and preferably it is 0.1-20%.

In the case that the dosage form of the medicinal composition of the present invention is a liquid or solution, the percentages of the active ingredients contained in the medicinal composition are, for example, usually 0.01-10 mg/mL for atorvastatin and pravastatin, and preferably 0.05-5 mg/mL. In the case of simvastatin, the percentage is usually 0.005-5 mg/mL, and preferably 0.03-3 mg/mL. Furthermore, the percentage of pyridoxine derivative is usually 0.1-20 mg/mL, and preferably 0.1-10 mg/mL.

### COMPOSITION EXAMPLES

The present invention will further be exemplified in more detail by the Examples. However, the scope of the present invention is not limited by these Examples.

### EXAMPLES

### Example 1

### Tablets

(1) Compositions

| | 6 Tablets | 6 Tablets | 6 Tablets |
|---|---|---|---|
| | (mg) | (mg) | (mg) |
| Pravastatin sodium | 20 | — | — |
| Simvastatin | — | 10 | — |
| Atorvastatin calcium | — | — | 20 |
| Pyridoxine hydrochloride | 100 | 100 | — |
| Pyridoxal phosphate | — | — | 60 |
| Magnesium oxide | 400 | 400 | 400 |
| Magnesium | 140 | 140 | 140 |
| aluminometasilicate | | | |
| Crystalline cellulose | 120 | 120 | 120 |
| Corn starch | 140 | 140 | 140 |
| Hydroxypropylcellulose | 60 | 60 | 60 |
| Croscarmellose sodium | 15 | 15 | 15 |
| Magnesium stearate | 25 | 25 | 25 |
| Glycerin triacetate | 6 | 6 | 6 |
| Lactose | A suitable | A suitable | A suitable |
| | amount | amount | amount |
| Total | 1,200 | 1,200 | 1,200 |

(2) Manufacturing Methods

Each active ingredient described above is weighed and the tablets are manufactured according to methods described in General Rules for Preparation (tablets) of the Japanese Pharmacopoeia.

### Example 2

### Granules

(1) Compositions

| | 3 Packages | 3 Packages | 3 Packages |
|---|---|---|---|
| | (mg) | (mg) | (mg) |
| Pravastatin sodium | 20 | — | — |
| Simvastatin | — | 10 | — |
| Atorvastatin calcium | — | — | 20 |
| Pyridoxine hydrochloride | 100 | 100 | — |
| Pyridoxal phosphate | — | — | 60 |
| Magnesium oxide | 400 | 400 | 400 |
| Magnesium | 140 | 140 | 140 |
| aluminometasilicate | | | |
| Purified sucrose | 1400 | 1400 | 1400 |
| Extracted products from stevia | 15 | 15 | 15 |
| Corn starch | 1200 | 1000 | 1100 |
| Polysorbate 80 | 80 | 80 | 80 |
| Magnesium stearate | 25 | 25 | 25 |
| Lactose | A suitable | A suitable | A suitable |
| | amount | amount | amount |
| Total | 4,300 | 4,300 | 4,300 |

(2) Manufacturing Methods

Each active ingredient described above is weighed and the granules are manufactured according to methods described in General Rules for Preparation (granules) of the Japanese Pharmacopoeia.

### Example 3

### Capsules

(1) Compositions

| | 6 Capsules | 6 Capsules | 6 Capsules |
|---|---|---|---|
| | (mg) | (mg) | (mg) |
| Pravastatin sodium | 20 | — | — |
| Simvastatin | — | 10 | — |
| Atorvastatin calcium | — | — | 20 |
| Pyridoxine hydrochloride | 100 | 100 | — |
| Pyridoxal phosphate | — | — | 60 |
| Magnesium oxide | 400 | 400 | 400 |
| Corn starch | 600 | 400 | 500 |
| Polysorbate 80 | 50 | 50 | 50 |
| Magnesium stearate | 25 | 25 | 25 |
| Lactose | A suitable | A suitable | A suitable |
| | amount | amount | amount |
| Capsule | 480 | 480 | 480 |
| Total | 2,300 | 2,300 | 2,300 |

(2) Manufacturing Methods

Each active ingredient described above is weighed and granules are manufactured according to methods described in General Rules for Preparation (granules) of the Japanese Pharmacopoeia. The capsules are manufactured by filling the granules in hard capsules.

### Example 4

### Syrups

(1) Compositions

| | 60 mL (mg) | 60 mL (mg) | 60 mL (mg) |
|---|---|---|---|
| Pravastatin sodium | 20 | — | — |
| Simvastatin | — | 10 | — |
| Atorvastatin calcium | — | — | 20 |
| Pyridoxine hydrochloride | 100 | 100 | — |
| Pyridoxal phosphate | — | — | 60 |
| Sodium benzoate | 240 | 240 | 240 |
| Citric acid | 60 | 60 | 60 |
| Sucrose | 1,500 | 1,500 | 1,500 |
| Conc. Glycerin | 1,800 | 1,800 | 1,800 |
| Polyvinylalcohol | 120 | 120 | 120 |
| Ethanol (95%) | 500 | 9,000 | 4,500 |
| Hydrochloric acid | A suitable | A suitable | A suitable |
| | amount | amount | amount |
| Sodium hydroxide | A suitable | A suitable | A suitable |
| | amount | amount | amount |
| Purified water | A suitable | A suitable | A suitable |
| | amount | amount | amount |

(2) Manufacturing Methods

Each active ingredient described above is weighed and the syrups are manufactured according to methods described in General Rules for Preparation (syrups) of the Japanese Pharmacopoeia. The syrups are kept in brown glass bottles.

### Example 5

### Tablets

(1) Compositions

| | 6 Tablets (mg) |
|---|---|
| Atorvastatin calcium | 20 |
| Pyridoxine hydrochloride | 100 |
| Magnesium oxide | 400 |
| Magnesium aluminometasilicate | 140 |
| Crystalline cellulose | 120 |
| Corn starch | 140 |
| Hydroxypropylcellulose | 60 |
| Croscarmellose sodium | 15 |
| Magnesium stearate | 25 |
| Glycerin triacetate | 6 |
| Lactose | A suitable amount |
| Total | 1,200 |

(2) Manufacturing Methods

Each active ingredient described above is weighed and the tablets are manufactured according to methods described in General Rules for Preparation (tablets) of the Japanese Pharmacopoeia.

### Example 6

### Granules

(1) Compositions

| | 3 Packages (mg) |
|---|---|
| Atorvastatin calcium | 20 |
| Pyridoxine hydrochloride | 100 |
| Magnesium oxide | 400 |
| Magnesium aluminometasilicate | 140 |
| Purified sucrose | 1400 |
| Extracted products from stevia | 15 |
| Corn starch | 1200 |
| Polysorbate 80 | 80 |
| Magnesium stearate | 25 |
| Lactose | A suitable amount |
| Total | 4,300 |

(2) Manufacturing Methods

Each active ingredient described above is weighed and the granules are manufactured according to methods described in General Rules for Preparation (granules) of the Japanese Pharmacopoeia.

### Example 7

### Capsules

(1) Compositions

| | 6 Capsules (mg) |
|---|---|
| Atorvastatin calcium | 20 |
| Pyridoxine hydrochloride | 100 |
| Magnesium oxide | 400 |
| Corn starch | 600 |
| Polysorbate 80 | 50 |
| Magnesium stearate | 25 |
| Lactose | A suitable amount |
| Capsule | 480 |
| Total | 2,300 |

(2) Manufacturing Methods

Each active ingredient described above is weighed and granules are manufactured according to methods described in General Rules for Preparation (granules) of the Japanese Pharmacopoeia. The capsules are manufactured by filling the granules in hard capsules.

### Example 8

### Syrups

(1) Compositions

| | 60 mL (mg) |
|---|---|
| Atorvastatin calcium | 20 |
| Pyridoxine hydrochloride | 100 |
| Sodium benzoate | 240 |
| Citric acid | 60 |
| Sucrose | 1,500 |
| Conc. Glycerin | 1,800 |
| Polyvinylalcohol | 120 |
| Ethanol (95%) | 500 |
| Hydrochloric acid | A suitable amount |
| Sodium hydroxide | A suitable amount |
| Purified water | A suitable amount |

(2) Manufacturing Methods

Each active ingredient described above is weighed and the syrups are manufactured according to methods described in General Rules for Preparation (syrups) of the Japanese Pharmacopoeia. The syrups are kept in brown glass bottles.

### Test Examples

### Test Example 1

(1) Test Substance

Pravastatin sodium manufactured at Sankyo Co., Ltd. was used. Simvastatin and atorvastatin synthesized at Chemtech Labo., Inc. were used. Pyridoxine hydrochloride manufactured at Nippon Roche K. K. was used.

(2) Animals

Male Beagle dogs of 5 months of age were purchased from Covance Research Products Inc. as the test animals and were used after accommodation breeding for approximately 5 months.

(3) Dosage Form, Preparation of the Formulation, and Storage

The required amount of the test substance calculated based on the body weight of the test animals was weighed and put in gelatin capsules (TORPAC Inc., ½ oz). After filling up, the capsules were placed in a box divided up for every animal and stored under freezing until use.

(4) Route of Administration and Administration Period

The capsules filled up with the test substance were orally administered to the test animals once daily via an oral gavage between 9:00-12:30. The test animals were fasted for 2-3 hrs prior to each administration. The administration period was 11 days.

(5) Preparation of the Test Samples

Approximately 10 mL of blood was collected from the cephalic vein of each dog on −14 and −7 day (the 1st and the 2nd week prior to administration) as well as 4, 8, and 12 days after administration. The animals were fasted for approximately 18 hrs before collection of the blood.

The collected blood was placed in a test tube and allowed to stand at room temperature for 30 min to 1 hour. Then the blood was centrifuged (approximately 1,600 g, for 10 min) and the resultant serum obtained was used for assays.

(6) Test Procedure

Total cholesterol content was assayed by enzymatic assay method, while HDL, LDL, and triglyceride were determined by homogeneous method, chemically modified method, and enzymatic techniques, respectively. Clinical Laboratory System (TBA-120FR, Toshiba Medical Systems Corporation) was used in all of these determinations.

(7) Results

Relative values of various levels of serum lipids in animals treated with each dose of pyridoxine hydrochloride, pravastatin sodium, simvastatin, or atorvastatin calcium alone as well as the medicinal compositions described above were calculated against each converted average value calculated from that determined 2 weeks and 1 week before the treatment into 100.

The results are summarized in Tables 1-6. The values indicate average results calculated from 5 dogs per a group.

TABLE 1

| | Percent changes of blood total | | |
|---|---|---|---|
| | cholesterol levels | | |
| | 4 Days | 8 Days | 12 Days |
| | after | after | after |
| Test substance (mg/Kg) | treatment | treatment | treatment |
| Simvastatin (1) | 94.8 | 94.1 | 92.4 |
| Pyridoxine hydrochloride (50) | 98.0 | 93.3 | 90.5 |
| Simvastatin (1) + Pyridoxine | 88.5 | 83.6 | 80.0 |
| hydrochloride (50) | | | |

TABLE 2

| | Percent changes of blood LDL levels | | |
|---|---|---|---|
| | 4 Days | 8 Days | 12 Days |
| | after | after | after |
| Test substance (mg/Kg) | treatment | treatment | treatment |
| Simvastatin (1) | 83.9 | 90.4 | 81.3 |
| Pyridoxine hydrochloride (50) | 101.7 | 95.0 | 91.4 |
| Simvastatin (1) + Pyridoxine | 83.0 | 70.4 | 70.4 |
| hydrochloride (50) | | | |

TABLE 3

| | Percent changes of blood | | |
|---|---|---|---|
| | triglyceride levels | | |
| | 4 Days | 8 Days | 12 Days |
| | after | after | after |
| Test substance (mg/Kg) | treatment | treatment | treatment |
| Simvastatin (1) | 66.4 | 85.3 | 82.0 |
| Pyridoxine hydrochloride (50) | 83.8 | 86.7 | 81.2 |
| Simvastatin (1) + Pyridoxine | 50.1 | 54.4 | 65.1 |
| hydrochloride (50) | | | |

TABLE 4

| | Percent changes of blood LDL levels | | |
|---|---|---|---|
| | 4 Days | 8 Days | 12 Days |
| | after | after | after |
| Test substance (mg/Kg) | treatment | treatment | treatment |
| Pravastatin sodium (2) | 92.6 | 91.6 | 90.4 |
| Pyridoxine hydrochloride (50) | 101.7 | 95.0 | 91.4 |
| Pravastatin sodium (2) + | 50.1 | 54.4 | 65.1 |
| Pyridoxine hydrochloride (50) | | | |

TABLE 5

| | Percent changes of blood LDL levels | | |
|---|---|---|---|
| | 4 Days | 8 Days | 12 Days |
| | after | after | after |
| Test substance (mg/Kg) | treatment | treatment | treatment |
| Atorvastatin calcium (2) | 82.4 | 82.1 | 83.6 |
| Pyridoxine hydrochloride (50) | 101.7 | 95.0 | 91.4 |
| Atorvastatin calcium (2) + | 76.1 | 73.8 | 66.2 |
| Pyridoxine hydrochloride (50) | | | |

TABLE 6

| | Percent changes of blood | | |
|---|---|---|---|
| | triglyceride levels | | |
| | 4 Days | 8 Days | 12 Days |
| | after | after | after |
| Test substance (mg/Kg) | treatment | treatment | treatment |
| Atorvastatin calcium (2) | 77.2 | 86.3 | 86.4 |
| Pyridoxine hydrochloride (50) | 83.8 | 86.7 | 81.2 |
| Atorvastatin calcium (2) + | 59.1 | 68.7 | 65.4 |
| Pyridoxine hydrochloride (50) | | | |

The various lipids in blood were remarkably reduced following combined administration of simvastatin, pravastatin, or atorvastatin with pyridoxine hydrochloride.

### Test Example 2

(1) Test Substance

Atorvastatin synthesized at Chemtech Labo., Inc. and pyridoxine hydrochloride manufactured at Nippon Roche K. K. were used.

(2) Animals

Male Beagle dogs of 5 months of age were purchased from Covance Research Products Inc. as the test animals and were used after accommodation breeding for approximately 5 months.

(3) Dosage Form, Preparation of the Formulation, and Storage

The required amount of the test substance calculated based on the body weight of the test animals was weighed and put in gelatin capsules (TORPAC Inc., ½ oz). After filling up, the capsules were placed in a box divided up for every animal and stored under freezing until use.

(4) Route of Administration and Administration Period

The capsules filled up with the test substance were orally administered to the test animals once daily via an oral gavage between 9:00-12:30. The test animals were fasted for 2-3 hrs prior to each administration. The administration period was 11 days.

(5) Preparation of the Test Samples

Approximately 10 mL of blood was collected from the cephalic vein of each dog on −14 and −7 day (the 1st and the 2nd week prior to administration) as well as 4, 8, and 12 days after administration. The animals were fasted for approximately 18 hrs before collection of the blood.

The collected blood was placed in a test tube and allowed to stand at room temperature for 30 min to 1 hour. Then the blood was centrifuged (approximately 1,600 g, for 10 min) and the resultant serum obtained was used for assays.

(6) Test Procedure

Blood homocysteine levels were determined by HPLC method used conventionally in current clinical examination.

(7) Results

Relative values of blood homocysteine levels in animals treated with each dose of atorvastatin calcium or pyridoxine hydrochloride alone as well as the medicinal compositions of atorvastatin calcium plus pyridoxine hydrochloride were calculated against each converted average value calculated from that determined 2 weeks and 1 week before the treatment into 100.

The results are summarized in Tables 7. The values indicate average results calculated from 5 dogs per a group.

TABLE 7

| | Percent changes of blood | | |
|---|---|---|---|
| | homocysteine levels (%) | | |
| | 4 Days | 8 Days | 12 Days |
| | after | after | after |
| Test substance (mg/Kg) | treatment | treatment | treatment |
| Atorvastatin calcium (2) + | 97.4 | 101.2 | 93.0 |
| Pyridoxine hydrochloride (5) | | | |
| Atorvastatin calcium (2) + | 90.8 | 89.0 | 85.7 |
| Pyridoxine hydrochloride (50) | | | |
| Comparative examples | | | |
| Atorvastatin calcium (2) | 107.2 | 106.3 | 106.9 |
| Pyridoxine hydrochloride (5) | 100.0 | 116.0 | 109.8 |
| Pyridoxine hydrochloride (50) | 101.4 | 111.5 | 103.0 |

Remarkable ameliorating effects on blood homocysteine levels appeared by combined administration of atorvastatin calcium with pyridoxine hydrochloride.

Since the medicinal compositions comprising an HMG-CoA reductase inhibitor and a pyridoxine derivative of the present invention exert potent mitigating effects on blood lipid levels and reduce blood homocysteine levels, the medicinal compositions of the present invention are considered useful as preventive or therapeutic agents against cardiovascular diseases such as hypercholesterolemia, arteriosclerosis, ischemic heart disease, myocardial infarction, thrombosis, peripheral vascular disease, Burger's disease, Raynaud's disease, and the like, cerebrovascular diseases such as cerebral infarction, cerebrovascular disorders, senile dementia, and the like, and neurological diseases such as Alzheimer's disease, Parkinson disease, and the like. Furthermore, the medicinal compositions of the present invention may also be useful as preventive or therapeutic agents against increased blood homocysteine levels caused by aging, smoking, nutrition impairment related to homocysteine metabolism, drugs, renal hypofunction, chronic renal dysfunction, diabetes mellitus, insulin resistance, malignant neoplasm, thyroidal hypofunction, pernicious anemia, and the like.

1. A medicinal composition comprising, in amounts effective for mitigating blood lipid levels or reducing high blood homocysteine levels,
an HMG-CoA reductase inhibitor and
a pyridoxine derivative in which the pyridoxine derivative is one or more pyridoxine derivatives selected from a group consisting of pyridoxine, pyridoxal, pyridoxamine, and their pharmacologically acceptable salts.

2. A medicinal composition according to claim 1, in which the HMG-CoA reductase inhibitor is one or more HMG-CoA reductase inhibitors selected from the group consisting of pravastatin, lovastatin, simvastatin, fluvastatin, rivastatin, atorvastatin, pitavastatin, and rosuvastatin.

3. A medicinal compositions according to claim 2, in which the HMG-CoA reductase inhibitor is pravastatin.

4. A medicinal composition according to claim 2, in which the HMG-CoA reductase inhibitor is simvastatin.

5. A medicinal composition according to claim 2, in which the HMG-CoA reductase inhibitor is atorvastatin.

6. A medicinal composition according to claims 1 or 2, in which the pyridoxine derivative is pyridoxamine or its pharmacologically acceptable salts.

7. A medicinal composition according to claims 1 or 2, in which the pyridoxine derivative is pyridoxal or its pharmacologically acceptable salts.

8. A medicinal composition according to claims 1 or 2, in which the pyridoxine derivative is pyridoxine or its pharmacologically acceptable salts.

9. A medicinal compositions according to claim 1, in which the HMG-CoA reductase inhibitor is atorvastatin or its salts and the pyridoxine derivative is pyridoxine or its pharmacologically acceptable salts.

10. A medicinal composition according to claim 1 in dosage form wherein the dosage form is a solid dosage form containing 0.01-5% atorvastatin or pravastatin, or 0.005-3% simvastatin, and 0.01-30% of pyridoxine derivative.

11. A medicinal composition according to claim 1 in dosage form wherein the dosage form is a liquid or solution dosage form containing 0.01-10 mg/ml of atorvastatin and pravastatin or 0.005-5 mg/ml simvastatin, and 0.1-20 mg/l of said pyridoxine derivative.

12. A method of mitigating blood lipid levels or reducing high blood homocysteine levels comprising administering in amounts effective for mitigating blood lipid levels or reducing high blood homocysteine levels, an HMG-CoA reductase inhibitor and a pyridoxine derivative in which the pyridoxine derivative is one or more pyridoxine derivatives selected from a group consisting of pyridoxine, pyridoxal, pyridoxamine, and their pharmacologically acceptable salts.

13. A method according to claim 12 comprising administering an HMG-CoA reductase inhibitor with a pyridoxine derivative at the same time or independently at a certain time interval.

14. A method according to claim 12 comprising mitigating blood lipid levels.

15. A method according to claim 12 comprising reducing high blood homocysteine levels.

16. A method according to claim 12, wherein the administering is to treat or prevent diseases caused by high blood homocysteine levels.

17. A method according to claim 12, wherein the administering is to prevent or treat hypercholesterolemia, arteriosclerosis, ischemic heart disease, myocardial infarction, thrombosis, peripheral vascular disease, Burger's disease, Raynaud's disease, cerebral infarction, cerebrovascular disorders, senile dementia, Alzheimer's disease, or Parkinson disease.

18. A method according to claim 12 comprising administering to an adult human 0.02 to 3 mg/kg per day of said HMG-CoA reductase inhibitors and 0.002 to 20 mg/kg per day of said pyridoxine derivative.

19. A method according to claim 18 wherein 0.08-2.5 mg/kg per day of said HMG-CoA reductase inhibitor an 0.02-13 mg/kg per day of said pyridoxine derivative are administered to the adult human.

20. A method according to claim 18 in which the HMG-CoA reductase inhibitor is one or more HMG-CoA reductase inhibitors selected from the group consisting of pravastatin, lovastatin, simvastatin, fluvastatin, rivastatin, atorvastatin, pitavastatin, and rosuvastatin.

21. A method according to claim 12 in which the HMG-CoA reductase inhibitor is one or more HMG-CoA reductase inhibitors selected from the group consisting of pravastatin, lovastatin, simvastatin, fluvastatin, rivastatin, atorvastatin, pitavastatin, and rosuvastatin.
